(19) United States
(12) Patent Application Publication
(10) Pub. No.: US 2009/0281567 A1
(43) Pub. Date: Nov. 12, 2009

(54) OCCLUSION ELEMENT FOR UNWANTED OPENINGS IN THE HEART

(75) Inventor: Peter Osypka, Rheinfelden-Herten (DE)

Correspondence Address: VOLPE AND KOENIG, P.C., UNITED PLAZA, SUITE 1600, 30 SOUTH 17TH STREET, PHILADELPHIA, PA, 19103, US

(73) Assignee: Peter Osypka Stiftung Stiftung des burgerlichen Rechts, Grenzach-Wyhlen (DE)

(21) Appl. No.: 12/436,841

(22) Filed: May 7, 2009

(30) Foreign Application Priority Data
May 7, 2008 (DE) 102008022673.4-35

(51) Int. Cl. A61B 17/08

(52) U.S. Cl. 606/215; 606/213

(57) ABSTRACT

An occlusion element (1) for closing an unwanted opening in the heart having two occlusion disks (3) formed from a braid of threads (2) is provided, wherein the threads (2) and the braid of these occlusion disks are each held by a sleeve or holder (5) and (6). The two occlusion disks (3) are initially separate and can be connected with their respective holders (5) and (6), which are oriented toward each other for forming the final shape of the occlusion element (1). The holders (5) and (6) form a spacing between the two occlusion disks (3) and a region (4) with reduced radial dimensions relative to the occlusion disks (3), and provide an arrangement where there are no projecting holders for the braid or threads on the outsides or end faces of the occlusion element (1) and the occlusion disks (3).

### CROSS-REFERENCE TO RELATED APPLICATIONS

This application claims the benefit of German Application No. DE 10 2008 022 673.4, filed May 7, 2008, which is incorporated herein by reference as if fully set forth.

### BACKGROUND

The invention relates to an occlusion element for unwanted openings in the heart for atrial-septal defects (ASD), wherein this element is formed or braided from threads made from biocompatible metal and/or plastic, from absorbable plastic, from memory material threads, from memory-plastic threads, from Nitinol wires, or from wires or threads sheathed with plastic. This occlusion element has two occlusion disks formed from the threads. Between these disks the occlusion element has a region with smaller radial dimensions that passes through the opening in the heart. In the position of use, the occlusion disks grip over the edges of the unwanted opening in the heart on both sides.

Such an occlusion element is known from DE 10 2006 040 415 B3 and has proven effective and has the advantage compared with an occlusion element according to DE 695 34 505 T2 that only one holder, which is formed as a sleeve and projects opposite the occlusion element on one side, is required for the threads and as a connection for a feed element with which the occlusion element can be brought and placed at its defined position.

From US 2003/0055455 A1, a comparable occlusion element is known in which two occlusion disks that can be connected and one insertion instrument for each of these occlusion disks are provided and the occlusion disks can be connected to each other in the interior of the heart with the help of coupling means, such as hooks and loops or magnets.

From US 2007/0179527 A1, a comparable occlusion element is known in which the occlusion disks have an umbrella-like construction and there is a mutual connection that allows one disk to shift one inside the other with a telescoping motion. Due to its stiffness, this connection makes the insertion and the handling more difficult.

### SUMMARY

Therefore, the invention is based on the objective of providing an occlusion element of the type noted above in which a projecting sleeve or holder can be avoided and nevertheless the simplest possible insertion into a heart can be performed.

To meet this objective, the occlusion element according to the invention provides that the two occlusion disks each have a holder that fixes the radially extending threads forming the disks on one side and that project from the occlusion disks, and the holders are connected to each other for forming a spacing between the two occlusion disks that forms the region with the reduced radial dimension in the position of use. The holder of one occlusion disk has an axial opening or recess that extends outward and in which a projection of the holder of the second occlusion disk can be inserted and connected in this position. In this manner, the projection of one holder fits with a positive fit, can be screwed, and/or can be welded into the opening or recess or depression of the other holder, and at least one of the holders has a coupling point or opening facing away from the connection point to the other holder for the detachable engagement of an insertion instrument, and the coupling point or opening has a thread to which a counter thread of the insertion instrument fits.

The occlusion element can be formed according to the invention from two initially individual disks that are connected to the holders which hold the braiding or the threads of these disks together, so that the holders come to lie between the two disks. That is, no disks require a holder that projects outward and that could negatively affect the blood flow in the heart atria. In this way, not only is a tension-resistant connection produced, but in the case of welding, a connection is produced that is no longer even detachable for the two disks and thus features correspondingly greater stability.

Furthermore, a detachable connection of the insertion instrument with the occlusion element is possible in a simple way. For example, the opening on the holder could have internal threading and the insertion instrument could have matching external threading at its distal end.

Here it is useful if the holder of at least one occlusion disk has a coupling point or opening that faces away from the connection point to the holder of the other occlusion disk and that is used for connecting to the feed instrument, wherein the threads or the mesh width of the braiding of the occlusion disk are open or leave a passage for the feed instrument.

The braiding of such an occlusion disk usually has a wide mesh and is pliant so that a feed instrument can be pushed through in a simple manner, in order to reach one of the holders located between the occlusion disks and here the holder that has the coupling point for connecting to this feed instrument. Thus, the occlusion element can be gripped in a known manner with a feed element and brought to its defined position, without a holder or sleeve having to project outward on one side of the occlusion element and possibly negatively affecting the blood flow in one of the atria. It is especially favorable if the holders of the two occlusion disks facing each other in the position of use are connected to each other in a load-resistant manner. Thus, on one hand they can easily withstand the forces during the implantation procedure and, on the other hand, the forces possibly occurring during use, without being able to be moved apart from each other.

An advantageous construction provides that the two disks are connected to each other elastically in the position of use, that is, the spacing can be changed somewhat against a spring force, in order to allow a tight contact of the two disks on both sides of a heart wall and in this way to be able to take into account heart walls of different thicknesses.

The axial opening or recess of the holder of one occlusion disk can have internal threading and the matching projection of the holder of the second occlusion disk can have a matching threaded piece or external threading for screwing the two holders together. Thus, through such screwing, the tension-resistant connection can be established and secured by additional welding.

The coupling point or opening for the insertion instrument could be provided on the holder that carries the projection for coupling with the holder of the other occlusion disk. Thus, this holder has sufficient material and substance, in order to be able to form an opening, while the other holder, on its side, already has a recess or depression, so that another recess for the insertion instrument would be possible only through a corresponding extension.

The outer contours of at least one of the occlusion disks could be circular or could have a shape deviating from a circle. The occlusion elements could also have peripheral contours adapted to different anatomical relationships.

A simple embodiment is produced when the two occlusion disks are the same size or especially when the occlusion disk to be arranged in the right atrium is smaller than the other occlusion disk. Above all, the mentioned alternative allows the different blood pressures in the two atria to be taken into account and the larger occlusion disk to be arranged in the left atria in which it must withstand a higher blood pressure.

A modified embodiment can provide that the occlusion disk for the right atrium has a small number of threads or wires compared with the occlusion disk for the left atrium. This is possible because the blood pressure in the right atrium is less than in the left atrium. Thus, the number of wires or threads for the right atrium can be reduced, that is, less metal is to be housed in the heart accordingly. Simultaneously, the insertion instrument, as a rule, a catheter, could have a narrower configuration.

Above all, for the combination of individual or several of the previously described features and measures, an occlusion element is produced for unwanted openings in the heart with two occlusion disks that are spaced apart from each other and that are each formed from threads or a braid. Between these disks there is a region of smaller cross section that is formed at least essentially from the two holders or sleeves, with which the threads or braids of the occlusion disks are held, so that no sleeves or holders are needed on the outer faces of the occlusion element facing away from each other. Furthermore, each occlusion disk can be produced individually, which leads to a simpler production or braiding process than if two occlusion disks are formed by a common braiding process.

### BRIEF DESCRIPTION OF THE SEVERAL VIEWS OF THE DRAWINGS

Below, an embodiment of the invention is described in greater detail with reference to the drawing. Shown in partially schematized representation are:

FIG. 1 is a view of an occlusion element according to the invention for unwanted openings in the heart with two occlusion disks that are spaced apart from each other and that are formed from braiding, wherein the braids of each disk are held by holders, wherein these holders of the two occlusion disks are turned toward each other and are connected to each other and an insertion instrument passes through the braid of one occlusion disk and is attached to one of these holders,

FIG. 2 is a view of the two occlusion disks of the occlusion element according to the invention before their mutual connection, and

FIG. 3 is a view of an occlusion disk and the sleeve-like holder holding together its braid.

### DETAILED DESCRIPTION OF THE PREFERRED EMBODIMENTS

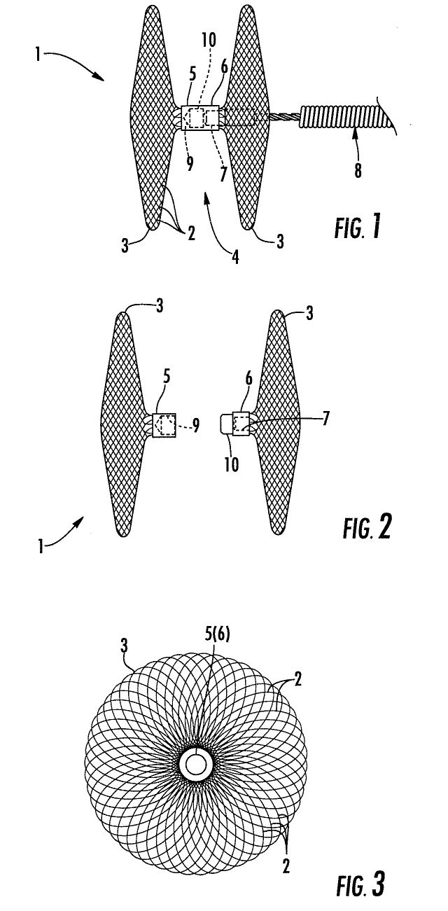

An occlusion element shown primarily in FIG. 1 and designated as a whole with 1 is illustrated and provided for insertion into unwanted openings in the heart as an ASD or occlusion instrument, but could also be used as a PDA or VSD occlusion device, as is known, for example, from DE 10 2006 040 415 B3 and other publications named therein.

This occlusion element 1 is formed from memory material threads 2, for example, from Nitinol threads or from wires or threads sheathed with plastic or also from memory plastic threads and advantageously braided, but could also be made from threads 2 made from biocompatible metal and/or plastic, e.g., from absorbable plastic. Above all, taking into account FIGS. 1 and 2 together with FIG. 3, it will become clear that, in the embodiment, these threads 2 form a braid.

According to FIG. 1, the occlusion element 1 has two occlusion disks 3 formed from the braid, and between these disks is a region 4 of smaller radial dimensions that passes through the opening in the heart in the position of use, wherein the occlusion disks 3 grip over the edges of the unwanted opening in the heart on both sides in the position of use and the region 4 is located in the opening. Here, in the position of use the occlusion disks 3 contact both sides of the wall of the heart having the opening and ensure that the unwanted opening is closed in a known manner.

With reference to FIG. 2, it becomes clear that both occlusion disks 3 have a holder 5 or 6. These holders fix the radially extending threads 2 forming these disks on one side and project from the occlusion disk 3. In addition, these holders 5 and 6 form a spacing between the two occlusion disks 3 according to FIG. 1 and form the region 4 with the reduced radial dimension in the position of use, when they are connected to each other in a way still to be described. Thus, an occlusion element 1 is produced in which the holders 5 and 6 for the braid of the occlusion disks 3 are not on their outsides, but instead, in the position of use, between the disks 3 and thus within the unwanted opening in the heart. Therefore, it is practically impossible to negatively affect the blood flow in the heart atria.

In FIGS. 1 and 2, it is illustrated that the holder 6 of one of the occlusion disks 3 has a coupling point or opening 7 that faces away from the connection point still to be explained for the holder 5 of the other occlusion disk 3. This coupling point or opening is used for connecting to an insertion instrument 8 indicated in FIG. 1, wherein the mesh width of the braid of the occlusion disk 3 opens or leaves a passage for this insertion instrument 8. In particular, the threads 2 of the braid are sufficiently flexible and pliant, in order to be able to insert the distal end of the insertion instrument 8 into the coupling point or opening 7.

According to FIG. 1, the holders 5 and 6 of the two occlusion disks 3 facing each other in the position of use are connected to each other in a load-resistant manner, in order to be able to be implanted and positioned with the help of the insertion instrument 8 and then, in the position of use, to be able to close the unwanted opening in the heart securely and without the risk of becoming separated from each other.

According to FIG. 2, it is provided that the holder 5 of one occlusion disk 3 has an axial opening or recess 9 that extends outward and in which a projection 10 of the holder 6 of the second occlusion disk 3 can be inserted and can be connected to this in this position, wherein FIG. 2 shows the still separated position and FIG. 1 shows the assembled position.

The projection 10 of the holder 6 here fits with a positive fit into the opening, recess or depression 9 of the other holder 5 and can thus be screwed and/or welded when the mutual connection is completed.

However, it is also conceivable that the axial opening or recess or depression 9 of the holder 5 of one occlusion disk 3 has internal threading and the matching projection 10 of the holder 6 of the other occlusion disk 3 has a matching threaded piece or external threading to allow screwing the two holders 5 and 6 together, which could be sufficient for mutual attachment, but could optionally also be secured by welding.

The holder 6 has the already mentioned coupling point or opening 7 on the side facing away from the connection point with the other holder 3 and provides, in the embodiment, a thread that is complementary to a counter thread on the distal end of the insertion instrument 8, wherein preferably the coupling point or opening 7 has internal threading and the insertion instrument 8 has matching external threading. Thus, the insertion instrument 8 can be coupled in a tension-resistant and compression-resistant way with the occlusion element 1 in the way shown in FIG. 1, in order to bring the occlusion element 1 into its position of use, after which the insertion instrument 8 can be unscrewed and removed again through opposite rotation.

The coupling point or opening 7 for the insertion instrument 8 is here provided on the holder that carries the projection 10 for coupling with the holder 5 and its opening or recess 9 of the other occlusion disk 3, because an additional coupling point or opening 7 on the holder 5 would not be possible without extending the holder 5 due to the mentioned opening or recess 9.

The outer contours of the occlusion disks 3 are circular in the embodiment according to FIG. 3, but could also have a shape that is different from a circle and, in contrast to the embodiment in which both occlusion disks 3 are of equal size, these could also have different sizes.

It should also be mentioned that instead of the direct connection of the two holders 5 and 6 like those provided in the embodiment, a connection that also has an intermediate piece would also be conceivable if the dimensions, on one hand, of the holders 5 and 6, and, on the other hand, of an opening to be closed in the heart makes this appear to be useful or necessary.

The occlusion element 1 is used for closing an unwanted opening in the heart and has, for this purpose, two occlusion disks 3 formed from a braid or threads 2, wherein the threads 2 and the braids of these occlusion disks are held by a sleeve or holder 5 and 6. The two occlusion disks 3 are here initially separated and can be connected with their two holders 5 and 6 pointing toward each other for forming the final shape of the occlusion element 1, so that the holders 5 and 6 form a spacing between the two occlusion disks 3 and a region 4 with radial dimensions that are reduced compared with the occlusion disks 3 arranged in parallel, so that there are no projecting holders for the braids or threads on the outsides or end faces of the occlusion element 1 and its occlusion disks 3 facing away from each other.

1. Occlusion element (1) for unwanted openings in the heart or for atrial-septal defects (ASD), comprising two occlusion disks (3) formed or braided from threads (2) made from at least one of biocompatible metal, biocompatible plastic, absorbable plastic, memory-material threads, memory-plastic threads, Nitinol wires, or wires or threads sheathed with plastic, a region (4) of reduced radial dimensions is defined between the disks (3) and is adapted to pass through the opening in the heart in the position of use and the occlusion disks (3) are adapted to grip over the edges of the unwanted opening in the heart on both sides in the position of use, the occlusion disks (3) each have a holder (5, 6) that fixes the threads (2) that extend radially and form the disks on one side that projects from the occlusion disk (3), the holders (5, 6) are connected to each other to form a space between the two occlusion disks (3) and form the region (4) with the reduced radial dimensions in the position of use, the holder (5) of a first one of the occlusion disks (3) has an axial opening, recess or depression (9) that faces outward and in which a projection (10) of the holder (6) of a second one of the occlusion disks (3) can be inserted and connected, the projection (10) of the holder (6) of the second occlusion disk is held with at least one of a positive fit, a threaded connection, or a welded connection in the opening, recess or depression (9) of the holder (5) of the first occlusion disk, at least one of the holders has a coupling point or opening (7) facing away from a connection point with the other holder (3) for the detachable engagement of an insertion instrument (8), and the coupling point or opening (7) has a complementary thread to a counter thread of the insertion instrument (8).

2. Occlusion element according to claim 1, wherein the holder (6) of at least one of the occlusion disks (3) has the coupling point or opening (7) facing away from the connection point to the holder (5) of the other occlusion disk (3) for connecting to the insertion instrument (8), and a width of the threads (2) or mesh of the braid of the occlusion disk (3) opens or leaves a passage for the insertion instrument (8).

3. Occlusion element according to claim 1, wherein the holders (5, 6) facing each other in the position of use of the two disks (3) are connected to each other in a load-resistant manner.

4. Occlusion element according to claim 1, wherein the axial opening, recess or depression (9) of the holder (5) of the first occlusion disk (3) has internal threading and the matching projection (10) of the holder (6) of the second occlusion disk (3) has a matching threaded piece or external threading for a mutual threaded engagement of the two holders (5, 6).

5. Occlusion element according to claim 1, wherein the coupling point or opening (7) for the insertion instrument (8) is provided on the holder (6) of the second occlusion disk that carries the projection (10) for coupling with the holder (5) of the first occlusion disk (3).

6. Occlusion element according to claim 1, wherein outer contours of at least one of the occlusion disks (3) is circular.

7. Occlusion element according to claim 1, wherein the two occlusion disks (3) are of equal size.

8. Occlusion element according to claim 1, wherein the occlusion disk (3) adapted for placement in the right atrium has a smaller number of threads (2) or wires than the occlusion disk (3) for the left atrium.

9. Occlusion element according to claim 1, wherein outer contours of at least one of the occlusion disks (3) is non-circular.

10. Occlusion element according to claim 1, wherein the occlusion disk (3) to be arranged in the right atrium is smaller than the other occlusion disk (3).
